(19) United States
(12) Patent Application Publication
(10) Pub. No.: US 2011/0040295 A1
(43) Pub. Date: Feb. 17, 2011

(54) Cancer treatment using selective photo-apoptosis

(75) Inventor: Brian Pierce, Chico, CA (US)

Correspondence Address: VAN PELT, YI & JAMES LLP, 10050 N. FOOTHILL BLVD #200, CUPERTINO, CA, 95014, US

(73) Assignee: Photometics, Inc.

(21) Appl. No.: 12/586,536

(22) Filed: Sep. 22, 2009

Related U.S. Application Data
(63) Continuation-in-part of application No. 12/034,022, filed on Feb. 20, 2008.
Application No. 12/034,022 is a division of application No. 10/789,948, filed on Feb. 26, 2004, now Pat. No. 7,354,433.
(60) Provisional application No. 60/450,736, filed on Feb. 28, 2003.

(51) Int. Cl. A61B 18/18

(52) U.S. Cl. 606/11

(57) ABSTRACT

A system for cancer treatment comprises a processor and a memory. The processor is configured to receive a target type and a host type and determine one or more illumination source characteristics such that: an illumination of the target type employing the one or more illumination source characteristics induces apoptosis in the target type without initiating thermolysis or ablation of the target type; and an illumination of the host type employing the one or more illumination source characteristics does not substantially induce apoptosis, thermolysis, or ablation in the host type. The memory is coupled to the processor and configured to provide the processor with instructions.

### CROSS REFERENCE TO OTHER APPLICATIONS

This application is a continuation in part of co-pending U.S. patent application Ser. No. 12/057,042 entitled “Differential Photochemical and Photomechanical Processing” filed Mar. 27, 2008, which is incorporated herein by reference for all purposes; which is a continuation of U.S. patent application Ser. No. 10/789,948 (Now U.S. Pat. 7,354,433), entitled DISINFECTION, DESTRUCTION OF NEOPLASTIC GROWTH, AND STERILIZATION BY DIFFERENTIAL ABSORPTION OF ELECTROMAGNETIC ENERGY filed Feb. 26, 2004, which is incorporated herein by reference for all purposes; which claims priority to U.S. Provisional Application No. 60/450,736, entitled DISINFECTION, DESTRUCTION OF NEOPLASTIC GROWTH, AND STERILIZATION BY DIFFERENTIAL ABSORPTION OF ELECTROMAGNETIC ENERGY filed Feb. 28, 2003 which is incorporated herein by reference for all purposes.

### BACKGROUND OF THE INVENTION

The development of cancer is associated with dysregulation of two common cell processes: proliferation and apoptosis. Common treatments for cancer prevent proliferation by eliminating or killing cancerous cells through surgical procedures or by using drugs or energy. Typical energy-based treatments of cancerous cells or tissues using heat or light target lysis (i.e., breaking open of an outer cell membrane), ablation (i.e., the use of heat to vaporize or eliminate), or excision (i.e., removal by a cutting process) of cancer cells or cancerous tissue. However, these treatments generally produce one or more undesired effects such as: energy damage to neighboring healthy cells or tissues, damage to neighboring healthy cells or tissues through the release of internal cellular components and treatment byproducts, wounds requiring healing, and necrotic tissue that must be absorbed by the body.

The body has a natural process for disposing cells that are no longer desired, apoptosis, that exhibits few or none of these undesired effects. When cells undergo the typical process of apoptosis, morphological alterations can be observed such as chromatin condensation, apoptotic body formation, phosphatidylserine translocation, or cellular shrinkage and blebbing prior to cell lysis. Following apoptosis, cells are typically phagocytosed by macrophages, parenchymal cells, or neoplastic cells and degraded within phogolysosomes without any essential inflammatory processes taking place in the surrounding tissue. Therefore, therapies that stimulate the apoptotic potential of cancer cells are generally less toxic to surrounding normal cells than therapies leading to necrosis, considered a toxic process that often affects large areas of cells and leads to inflammation from cellular destruction.

Attempts to create a cancer treatment that stimulate apoptosis have until now relied on the use of chemicals, drugs, or genetic manipulation.

### BRIEF DESCRIPTION OF THE DRAWINGS

Various embodiments of the invention are disclosed in the following detailed description and the accompanying drawings.

FIG. 1 is a block diagram illustrating an embodiment of a system for cancer treatment using selective photo-apoptosis.

FIG. 2 is a block diagram illustrating an embodiment of a source.

FIG. 3 is a block diagram illustrating an embodiment of a coupler.

FIG. 4 is a block diagram illustrating an embodiment of a head.

FIG. 5 is a block diagram illustrating an embodiment of a controller.

FIG. 6 is a diagram illustrating an embodiment of a scan pattern.

FIG. 7 illustrates example spectra of a target and a host in one embodiment.

FIGS. 8-11 are tables illustrating embodiments of wavelengths to target for treatment.

FIG. 12 is a flow diagram illustrating an embodiment of a process for a system for selective photo-apoptosis cancer treatment.

FIG. 13 is a flow diagram illustrating an embodiment of a process for a system for selective photo-apoptosis cancer treatment.

FIG. 14 is a flow diagram illustrating an embodiment of a process for a system for selective photo-apoptosis cancer treatment.

FIG. 15 is a flow diagram illustrating an embodiment of a process for determining a selective photo-apoptosis treatment.

FIGS. 16A and 16B are flow diagrams illustrating embodiments of processes for a system for selective photo-apoptosis cancer treatment.

FIG. 17 is a flow diagram illustrating an embodiment of a process for a system for selective photo-apoptosis cancer treatment.

FIG. 18 is a flow diagram illustrating an embodiment of a process for a system for selective cancer treatment.

### DETAILED DESCRIPTION

The invention can be implemented in numerous ways, including as a process; an apparatus; a system; a composition of matter; a computer program product embodied on a computer readable storage medium; and/or a processor, such as a processor configured to execute instructions stored on and/or provided by a memory coupled to the processor. In this specification, these implementations, or any other form that the invention may take, may be referred to as techniques. In general, the order of the steps of disclosed processes may be altered within the scope of the invention. Unless stated otherwise, a component such as a processor or a memory described as being configured to perform a task may be implemented as a general component that is temporarily configured to perform the task at a given time or a specific component that is manufactured to perform the task. As used herein, the term ‘processor’ refers to one or more devices, circuits, and/or processing cores configured to process data, such as computer program instructions.

A detailed description of one or more embodiments of the invention is provided below along with accompanying figures that illustrate the principles of the invention. The invention is described in connection with such embodiments, but the invention is not limited to any embodiment. The scope of the invention is limited only by the claims and the invention encompasses numerous alternatives, modifications and equivalents. Numerous specific details are set forth in the following description in order to provide a thorough understanding of the invention. These details are provided for the purpose of example and the invention may be practiced according to the claims without some or all of these specific details. For the purpose of clarity, technical material that is known in the technical fields related to the invention has not been described in detail so that the invention is not unnecessarily obscured.

A cancer treatment described as “selective photo-apoptosis” is disclosed. A target type (e.g., a cancerous tissue or cell) is received. A set of illumination source characteristics are determined such that an illumination of the target type by a source having the illumination source characteristics induces apoptosis in the target type without initiating thermolysis or ablation.

In some embodiments, both a target type and a host type (e.g., a healthy nearby tissue or cell) are received. A treatment is determined that uses illumination source characteristics that have the desired effect on the target type but do not substantially induce apoptosis, thermolysis, nor ablation in a host type. This “selective treatment” allows illumination with the same illumination source characteristics to be directed on an area that includes both target type and host type with the treatment occurring only in the target type.

In some embodiments, the selective photo-apoptosis treatment includes a scan pattern whereby an area larger than the spot associated with the illumination is used to deposit energy in the larger area presumably enabling a larger area of target type tissue to be treated.

In various embodiments, a target type and a host type are received by using an identifying spectrograph at different physical locations, by using visual inspection of an expert, by using a biopsy, by using a user interface, or any other appropriate manner of receiving a host type and target type. The spectra of the host and target are determined (e.g., using a measurement or by using predetermined reference spectra). In some embodiments, differences in the spectral properties of the host and target are used to determine the one or more wavelengths that will achieve the desired effect or effects in target types without disrupting host types.

In various embodiments, the delivery characteristics of the energy source, including pulse characteristics such as pulse width, pulse intensity, and number of pulses; scan characteristics such as pattern, scan rate change, scan skipping, a scan area, or scan speed(s); and other means for controlling thermal accumulation and achieving desired results are adjusted to enhance the effectiveness of the selective photo-apoptosis treatment.

In various embodiments, the one or more wavelengths determined for photo-apoptosis treatment break bonds in molecules, proteins, lipids, cellular structures, nuclei structures, or any other appropriate cell or tissue component. In some embodiments, multiple absorption bonds in a compound are targeted to achieve a desired, efficient, or required energy transfer.

In various embodiments, target cells or tissues comprise skin conditions such as actinic keratosis, basal cell carcinoma, dermatofibroma, dysplastic cevus, keratoacanthoma, lentigines, lentigo maligna, melanocytic nevi, melanoma of various types including acral lentiginous, amelanotic, desmoplastic, lentigo maligna, mucosal, nodular, polypoid, and soft-tissue, nevus composites, nevus intradermalis, seborrheic keratosis, squamous cell carcinoma, etc., and other appropriate skin conditions, or any other appropriate target cells or tissues.

In various embodiments, target cells or tissues comprise neo-plastic tissue such as prostate cancer, breast cancer, or any other neo-plastic conditions that can receive light energy directly or through a probe, guide, or other transmitting device, or any other appropriate target cells or tissues.

In some embodiments, a low power test treatment is made to test treatment dosimetry (treatment plan and illumination source characteristics). The results of the test treatment are measured and the results are used to adjust the treatment plan. In some embodiments, a measurement is made after treatment to determine effectiveness and used to determine whether additional treatment is necessary.

In some embodiments, multiple wavelengths are used to enhance effectiveness. A threshold for achieving desired effects in the target type and a threshold for disrupting a host type are determined. A selective photo-apoptosis treatment is determined having a plurality of wavelengths that together achieve the threshold for the desired effects in the target type without achieving the threshold for disruption of the host type. In some embodiments, at least two of the multiple wavelengths are selected such that they are used in combination to achieve a desired penetration depth. In some embodiments, a wavelength that has been determined for selective photo-apoptosis therapy is limited in its ability to penetrate the target and host types so a wavelength that has a greater ability to penetrate the target and host types is also used, thereby increasing the depth at which the first wavelength is able to achieve the threshold for achieving desired effects.

In some embodiments with multiple wavelengths, at least two of the multiple wavelengths are selected such that they are absorbed in a common bond site. In some embodiments, at least two of the multiple wavelengths are selected such that they comprise a fundamental absorption wavelength and a related overtone. In some embodiments, each wavelength will have a different scan pattern and other characteristics, including scan rate change, scan skipping, a scan area, or scan speed(s) to control thermal accumulation. In some embodiments, at least two of the multiple wavelengths are used to affect broad differential absorption peaks.

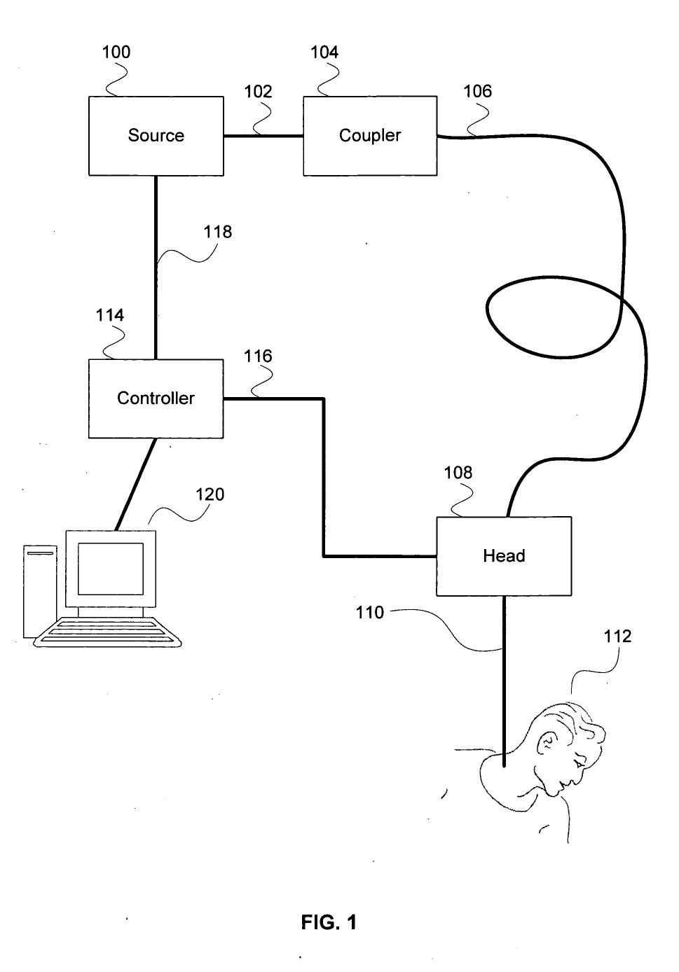

FIG. 1 is a block diagram illustrating an embodiment of a system for cancer treatment using selective photo-apoptosis. In the example shown, source 100 generates light at one or more specified wavelengths. For example, a coherent infrared light source with the ability to adjust: intensity, pulse width, wavelength, pulse sequence, etc. Source 100 provides its output along path 102 to coupler 104. Coupler 104 couples source 100 output to fiber 106. In various embodiments, coupler 104 comprises a light pipe or other conveyance method, a free beam, or any other appropriate coupling mechanism. Fiber 106 is coupled to head 108. Head 108 directs multiple wavelengths along path 110. For example, head 108 directs multiple wavelengths through an optical gel interface to a target 112 (e.g., face, trunk, skin, cells, tissue, etc.). In various embodiments, head 108 includes optics for generating a scan pattern, a detector for monitoring treatment effectiveness (e.g., the ability to measure a spectrograph of target 112 or detect a heat signature unique to the desired effect), optics for visually monitoring target 112, or any other appropriate components. Head 108 and source 100 receive control signal(s) from controller 114 using connector 116 and connector 118. Head 108 also provides controller 114 with data (e.g., from detector or visual monitoring). A user interfaces with controller 114 (e.g., using computer system 120) to control the system—for example, to indicate a target type and/or a host type, to view the target, to view the host, to start and/or stop a treatment, etc.

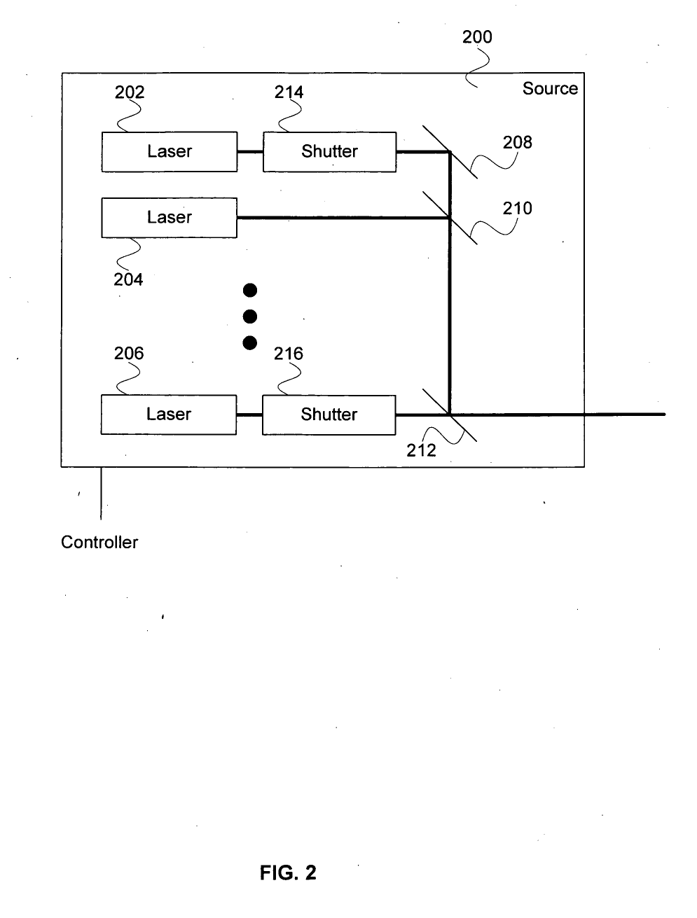

FIG. 2 is a block diagram illustrating an embodiment of a source. In some embodiments, source 200 is used to implement source 100 of FIG. 1. In the example shown, source 200 includes one or more light generators (e.g., laser 202, laser 204, laser 206, etc.) and combining optics (e.g., beam splitters 208, beam splitter 210, and beam splitter 212). In various embodiments, a light generator comprises a laser, a diode laser, a pumped laser and a harmonic generator, a ring laser, an incoherent light source, a flash lamp, or any other appropriate generator. Laser 202 and laser 206 each have a shutter (e.g., shutter 214 and shutter 216, respectively) to enable generation of specific pulse widths or to allow a number of pulses of a pulse train through. Laser 204 is a switched laser that is able to be controlled to produce one or more pulses (e.g., a diode laser). In various embodiments, source 200 includes polarization optics and/or filters for intensity control. In various embodiments, a light generator has an adjustable wavelength and/or produces multiple wavelengths. Source 200 is connected to a controller—for example, for setting intensity, pulse length, polarization, wavelength, etc. In some embodiments, one or more of the laser sources delivers background energy.

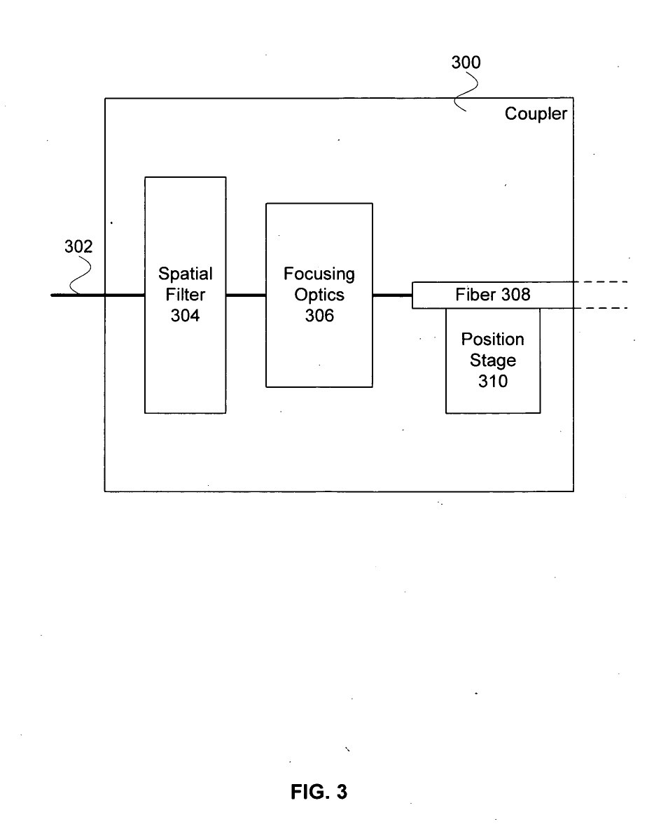

FIG. 3 is a block diagram illustrating an embodiment of a coupler. In some embodiments, coupler 300 is used to implement coupler 104 of FIG. 1. In the example shown, coupler 300 includes optics (e.g., spatial filter 304, focusing optics 306, and position stage 310) to couple input beam 302 with fiber 308. Input beam 302 is processed by spatial filter 304 and focused using focusing optics 306 to provide for efficient coupling to fiber 308. Fiber 308 is positioned for efficient coupling using position stage 310.

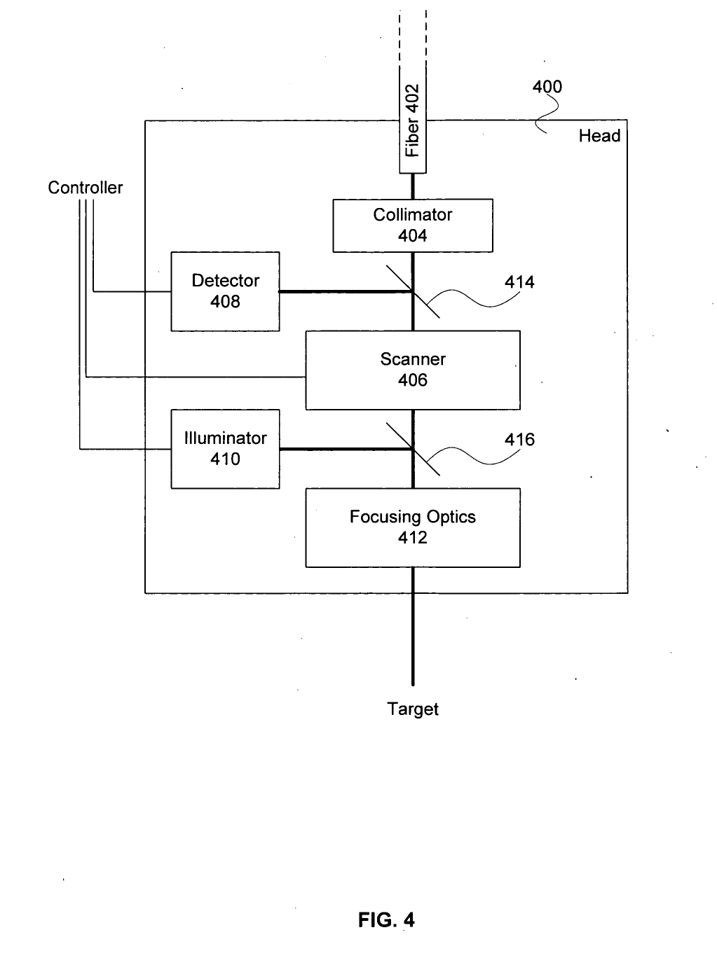

FIG. 4 is a block diagram illustrating an embodiment of a head. In some embodiments, head 400 is used to implement head 108 of FIG. 1. In the example shown, head 400 includes optics (e.g., collimator 404, scanner 406, focusing optics 412) to deliver the beam from fiber 402 to a target. Input beam from fiber 402 is processed by collimator 404, scanner 406, and focusing optics 412. In various embodiments, focusing optics 412 comprises one or more of the following: standard confocal, stereo lithography, or beam shaping. Head 400 includes illuminator 410 for illuminating the target for viewing the target or for providing a general delivery of energy to the target. Head 400 includes detector 408 for measuring target. (e.g., a spectrographic measurement) or for viewing target (e.g., imaging). Head 400 is connected to controller to control scanner 406 (e.g., to set scan pattern, scan rate, etc.), control illuminator 410, and receive data from detector 408. In some embodiments, illuminator 410 comprises one or more illuminators where one of the one or more illuminators delivers background energy to a target.

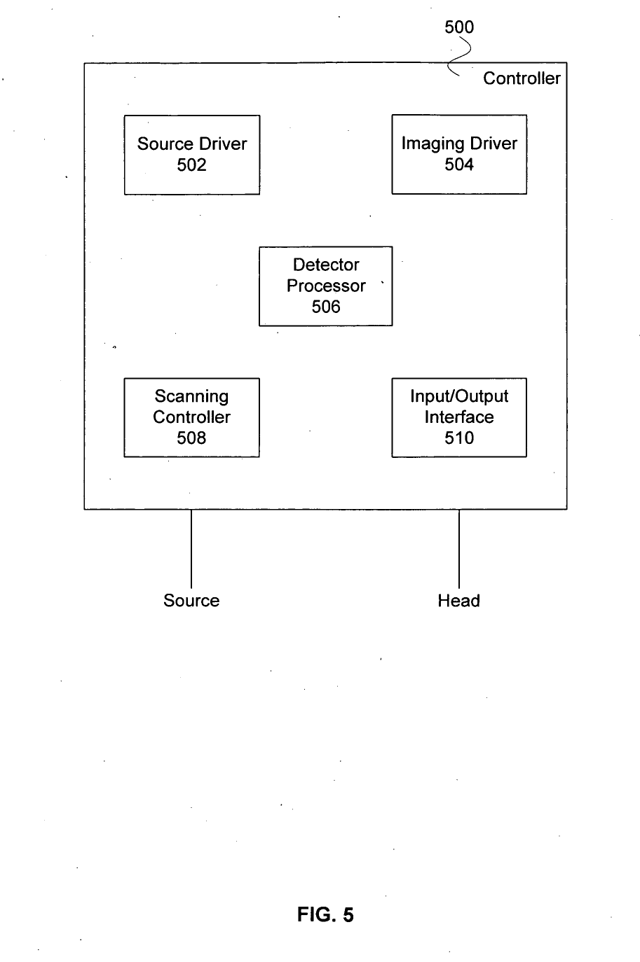

FIG. 5 is a block diagram illustrating an embodiment of a controller. In some embodiments, controller 500 is used to implement controller 114 of FIG. 1. In the example shown, controller 500 comprises source driver 502, imaging driver 504, detector processor 506, scanning controller 508, and input/output interface 510. Source driver 502 controls sources for the system including setting source wavelengths, intensities, pulse lengths, pulse sequences, etc. Imaging driver 504 controls receiving imaging data from a head, providing a user display of appropriate information, and providing associated controls for a user or for the imaging system including setting magnification, background illumination, panning, etc. Detector processor 506 controls receiving detector data from a head, providing a user display or processed detector information (e.g., spectrographs, etc.), and providing associated controls for a user or for the detector system including setting frequency of measurement, range of measurement, etc. Scanning controller 508 controls providing scanning for a head—for example, scan rate, scan pattern, etc. Input/output interface 510 receives information from and provides information to a user via a user interface system.

In various embodiments, controller 500 is implemented using one or more hardware processors and one or more software modules. In various embodiments, controller 500 includes semiconductor memory (e.g., random access memory, read only memories, etc.), magnetic memories (e.g., hard drives, redundant arrays of drives, etc.), or any other appropriate memories.

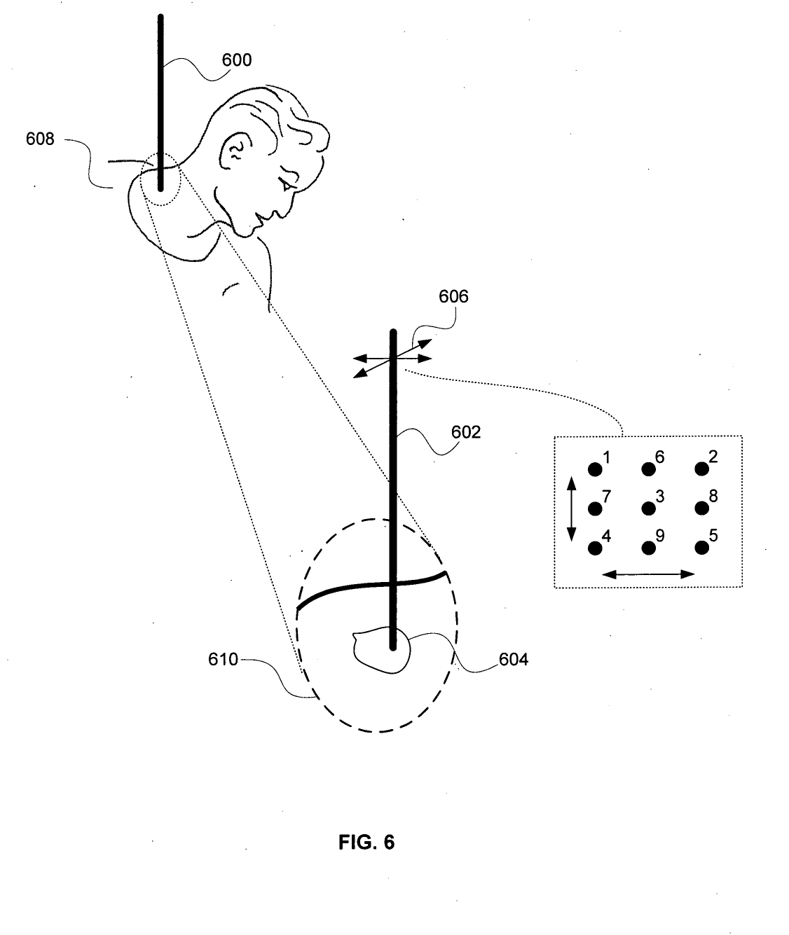

FIG. 6 is a diagram illustrating an embodiment of a scan pattern. In some embodiments, a scan pattern is set using scanning controller 508 of FIG. 5. In the example shown, head output beam 600 is incident on a target and host (e.g., torso 608). Blow up 610 illustrates head output beam 602 that is scanned in both directions perpendicular to the direction of the head output beam (e.g., orthogonal pattern 606 showing a skipping pattern with three rows 1, 6, 2; and 7, 3, 8; and 4, 9, 5) to illuminate target 604. In some embodiments, the skipping scan pattern is selected to reduce local heating (e.g., to protect host tissue from being damaged).

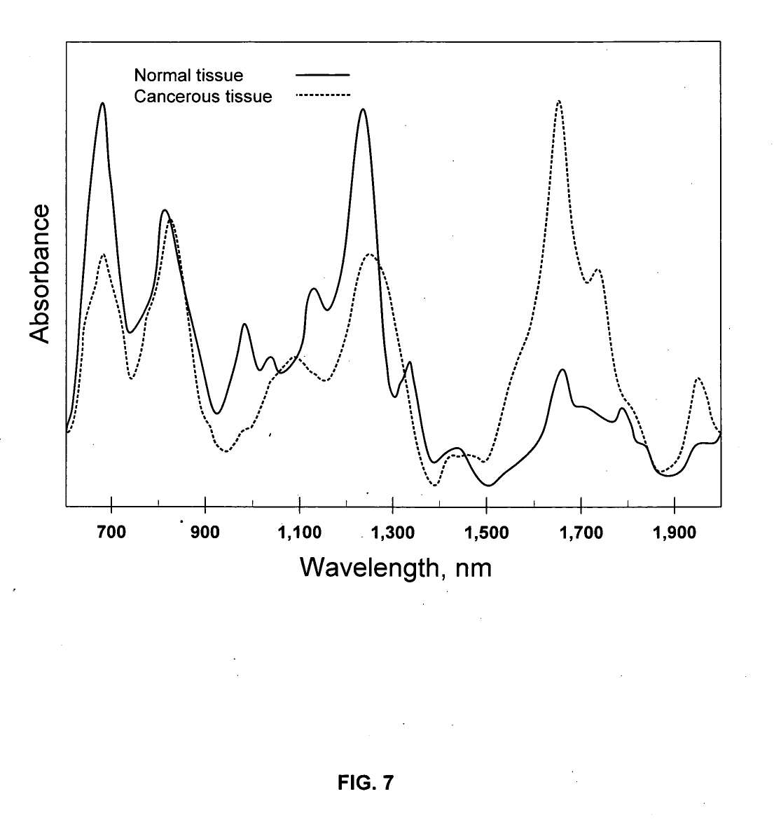

FIG. 7 illustrates example spectra of a target and a host in one embodiment. In the example shown, absorbance spectra are shown for normal (host) and cancerous (target) tissue for incident light with wavelengths from 700 nm to 1900 nm. Note that for the wavelengths around 1650 nm to 1700 nm, the absorbance of cancerous tissue is substantially higher than that of normal tissue. These wavelengths can be used for selective treatments of cancerous tissue over normal tissue.

In some embodiments, the identification of characteristics in target type and host type tissues appropriate for selective photo-apoptosis treatment starts with a comparison of the molecular structure of normal skin with the molecular structure of benign and malignant skin lesions using infrared Fourier or Ramen spectroscopy. Differences are found in the primary and secondary structure of proteins as reflected by the amide vibrations of peptide bonds which are characterized by a fundamental frequency with one or more overtones at higher frequencies. Variations in photo-mechanical responses may also be found among the principal lipid types, for examples in twisting versus wagging and in CH₂ and CH stretching vibrations. Histologically distinguishable lesions showed specific combinations of band changes indicating alterations in the protein confirmation and in the molecular structure of the lipids. As another example, histogenetically related lesions (e.g., actinic keratosis and squamous cell carcinoma) produced similar but not identical patterns of spectral changes.

The resulting spectra are evaluated to determine the set of various wavelengths that are absorbed by the target or cancer cells more readily than by the host or healthy cells. Since biomolecules generally have a very large number of accessible vibrational states, biomolecules unique to the target type tissue will generally manifest in multiple spectral differences. These differences are evaluated by their potential to disrupt cellular constituents in the target type tissue and therefore either promote or un-inhibit progression in apoptotic pathways through selective absorption of energy. Those areas of difference used for greatest selective effect are not necessarily those areas of maximum difference. Other characteristics, such as heat transfer properties of tissue, scattering coefficients, and variations in tissue opacity are factors that are considered.

There are many pathways leading to apoptotic induction. The intrinsic apoptotic pathway involves mitochondrial membrane permeabilization, release of cytochrome c into the cytosol, apoptosome formation, and activation of caspase-9 and down-stream caspases, leading to DNA fragmentation. The extrinsic apoptotic pathway is triggered through the activation of death receptors such as TNF-a (tumor necrosis factor-α) or Fas ligand on the cell membrane and can activate caspases partially independent of the mitochondria. Another pathway is granzymatic attack from immune cells involving caspase triggering granzyme B and caspase independent granzyme A. Major regulatory bottlenecks in apoptotic function revolve around the “execution pathway” initiator caspase 3, and the SET complex inhibition of DNAse NM23-H1. These pathways are by no means inclusive, as new mechanisms and contributing cellular factors are constantly being discovered.

In some embodiments, apoptosis is stimulated by disrupting cellular metabolism rather than targeting a specific compound in the apoptotic pathway. In some embodiments, targets for selective photo-apoptosis include organic compounds, particularly proteins, nucleic acids, polysaccharides, and lipids, which contribute to many metabolic processes that are interdependent and complex and that are essential to the viability of cells. Interrupting or diminishing one or more of these functions will often result in the destruction of the cell. Disrupting cellular redox balance or overproduction of reactive oxygen species often triggers apoptosis. Proteins are of particular importance among organic compounds and are thus the focus of some embodiments of selective photo-apoptosis treatment. The amino acid sequence and the three-dimensional conformation of a protein are critical to the biochemical function of a protein and its interactions with biological systems. Alterations in the three-dimensional conformation can result in deactivation of the protein and prevention of its ability to take part in biochemical processes. Each of these compounds and the associated bonds are potential targets for selective photo-apoptosis treatment.

In some embodiments, the proteins that are targeted are denatured. The denaturation of a protein is any non-covalent change in the structure of the protein. Denaturation typically alters the secondary, tertiary or quaternary structure of the protein, causing the protein to lose its biological activity. Denaturation of an enzyme results in the loss of enzymatic activity. One cause of denaturation is heat, and depending on the protein and on the severity of the heating, the denaturation and loss of activity can be reversible or irreversible. As the temperature is raised, changes to the protein occur progressively. The first changes are to the long-range interactions that are needed to maintain the tertiary structure. The interactions are weakened and then broken, resulting in a more flexible structure and in greater exposure of the protein to solvent. With increased heating, the cooperative bonds or interactions that stabilize the structure are affected, allowing water to interact with the amide nitrogen atoms and carbonyl oxygen atoms and to form new hydrogen bonds. The increased access of water also weakens nearby hydrogen bonds by increasing the effective dielectric constant near those bonds. This results in the exposure of hydrophobic groups to the solvent.

The exposure of hydrophobic groups and new hydrogen bonding groups to the water results in an increase in the amount of water bound by the protein molecule, which causes the protein to unfold. This unfolding increases the hydrodynamic radius of the molecule which in turn increases the viscosity of the solution. The protein will then attempt to minimize its free energy by burying hydrophobic groups while exposing polar groups to the solvent. While this is analogous to the original folding that occurred when the protein was first formed, this new rearrangement occurs at a much higher temperature, which greatly weakens the short-range interactions that initially direct protein folding. In addition many proteins are formed utilizing the free energy reduction of other enzymes and chaperone folding mechanisms not available after thermal denaturation. The resulting structure is often vastly different from that of the native protein and therefore prevents the protein from performing its function.

As heat-denatured proteins are cooled, the molecules are frequently not in a conformation having the lowest free energy and tend to aggregate through hydrophobic bonds, which create kinetic barriers that prevent the molecules from returning to their native conformation. Before the protein can re-fold and return to its native conformation, these hydrophobic bonds would first have to be dissociated, an event that is energetically unfavorable because of the exposure of large number of hydrophobic groups on the protein to the solvent. This transformation of the protein to a form in which it cannot re-fold and therefore cannot perform its biological function is a desired effect in the disruption of the biochemical process that is integral to the development and proliferation of cells.

In some embodiments, the selective photo-apoptosis treatment includes the determination of appropriate laser parameters such as wavelength, power density, exposure time, spot size, focal point, and repetition rate that are carefully matched with optical tissue properties like absorption, scattering coefficients, heat transfer, absorption coefficient, heat capacity and thermal conductivity to create the desired effect.

FIGS. 8-11 are tables illustrating embodiments of wavelengths to target for treatment. In some embodiments, wavelengths illustrated in tables 8-11 are used as the multiple wavelengths for a treatment of a target. In the example shown, the table in FIG. 8 comprises the wavelengths of absorbers involving Nitrogen and Hydrogen, which are found in the amino acid building blocks of proteins. The table in FIG. 9 comprises wavelengths of absorbers involving Carbon-Nitrogen bonds, which are found in the amino acid building blocks of proteins. The table in FIG. 10 comprises wavelengths of absorbers involving Carbon-Oxygen bonds, which are found in the amino acid building blocks of proteins. The table in FIG. 11 comprises Oxygen-Hydrogen bonds, which are found in the amino acid building blocks of proteins.

FIG. 12 is a flow diagram illustrating an embodiment of a process for a system for selective photo-apoptosis cancer treatment. In some embodiments, the process of FIG. 12 is implemented using controller 500 of FIG. 5. In the example, shown, in 1200 a target type and host type are received. For example, target type and host type are input on a user interface or automatically determined by the system using a measurement (e.g., a spectrograph or image analysis). In various embodiments, the target type and host type are identified using one or more of the following: a spectrograph of different locations, a visual inspection by an expert (e.g., a doctor, etc.), a biopsy, a user interface, a measurement using a detector on a head, or any other appropriate manner of identification and provided to the system. In 1202, one or more illumination source characteristics are determined such that an illumination of a target type by an illumination source with the illumination source characteristics will induce apoptosis but neither thermolysis nor ablation in the target type and an illumination of the host type by an illumination source with the illumination source characteristics does not substantially induce apoptosis in the host type.

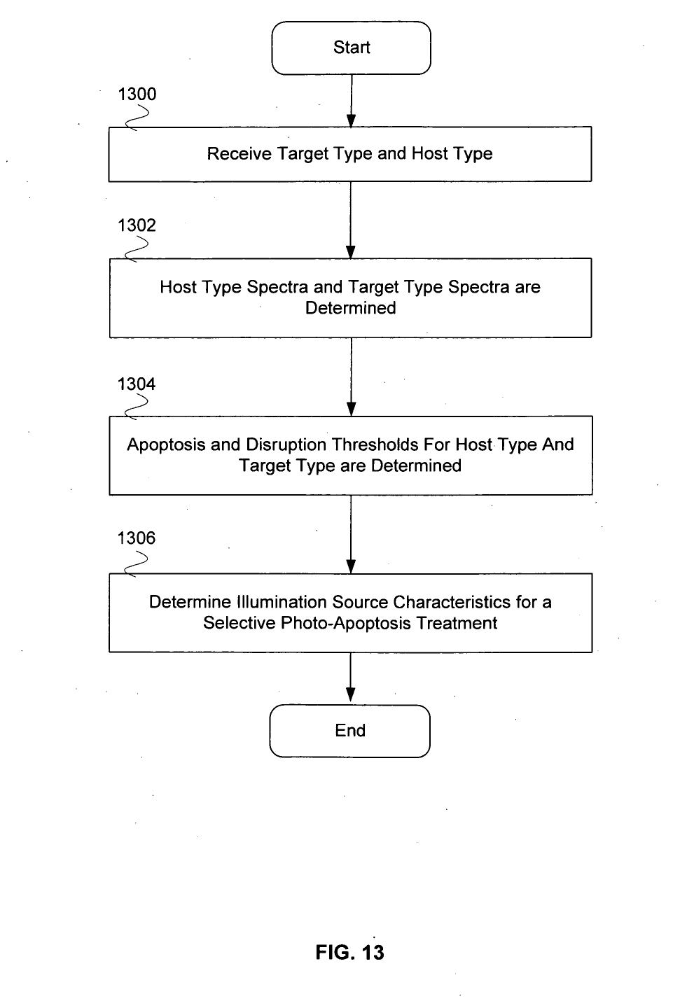

FIG. 13 is a flow diagram illustrating an embodiment of a process for a system for selective photo-apoptosis cancer treatment. In some embodiments, the process of FIG. 13 is implemented using controller 500 of FIG. 5. In the example, shown, in 1300 a target type and host type are received. For example, the host and target types are input on a user interface or automatically determined by the system using a measurement (e.g., a spectrograph or image analysis). In various embodiments, the target type and host type are identified using one or more of the following: a spectrograph of different locations, a visual inspection by an expert (e.g., a doctor, etc.), a biopsy, a user interface, a measurement using a detector on a head, or any other appropriate manner of identification and provided to the system. In 1302, a target type spectrum and a host type spectrum are determined. For example, the spectra are measured directly or are retrieved from a stored database. In 1304, a threshold for treatment of target type and threshold for disruption of host type are determined. For example, a differential calorimeter measurement, a spectrographic measurement, a reference spectrum, or other reference values or tables are used to determine treatment and disruption thresholds. In 1306, a selective photo-apoptosis treatment is determined. For example, a pattern for scanning is determined over a target volume for a sequence of pulses, where the pulse lengths and intensities are specified. In various embodiments, the wavelength(s) and/or focal properties of the head are selected to address the source generated light on a target volume.

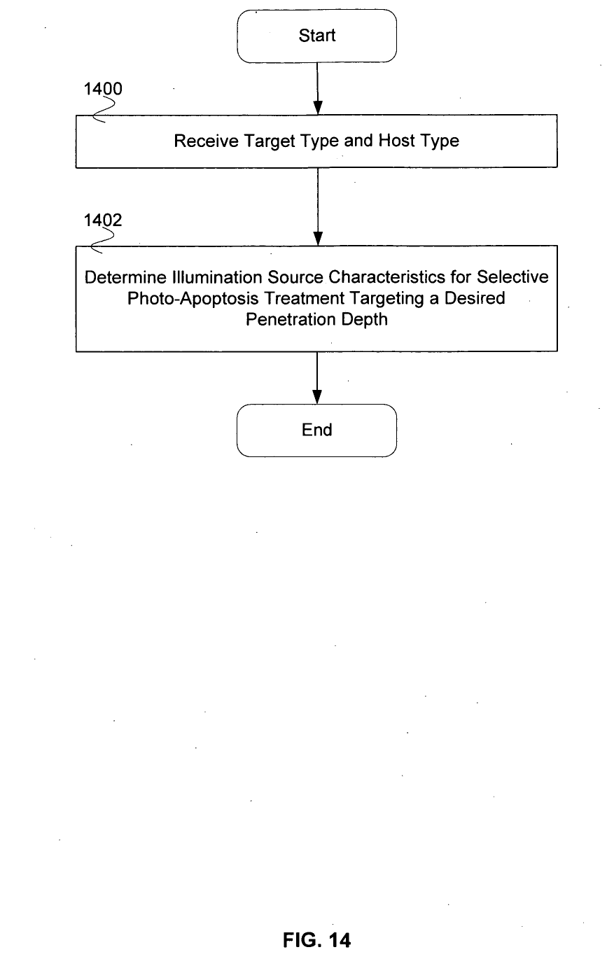

FIG. 14 is a flow diagram illustrating an embodiment of a process for a system for selective photo-apoptosis cancer treatment. In some embodiments, the process of FIG. 14 is implemented using controller 500 of FIG. 5. In the example, shown, in 1400 a target type and host type are received. For example, the host and target types are input on a user interface or automatically determined by the system using a measurement (e.g., a spectrograph or image analysis). In various embodiments, the target type and host type are identified using one or more of the following: a spectrograph of different locations, a visual inspection by an expert (e.g., a doctor, etc.), a biopsy, a user interface, a measurement using a detector on a head, or any other appropriate manner of identification and provided to the system. In 1402, one or more illumination source characteristics are determined to provide a selective photo-apoptosis treatment that targets a desired penetration depth. In various embodiments, the penetration depth is adjusted by choosing a different wavelength that is more or less able to penetrate the host types and target types, by changing pulse length, intensity, frequency, etc., by adjusting focal length or other delivery characteristics, by adding a second wavelength with a different ability to penetrate host types and target types, or by any other appropriate means of influencing penetration depth. In some embodiments, the one or more illumination source characteristics determined for the selective photo-apoptosis treatment at a desired penetration depth are not the one or more illumination source characteristics most effective for a selective photo-apoptosis treatment when penetration depth is not considered.

Selective photo-apoptosis avoids the typical effects of laser therapies: ablation or thermolysis. With ablation, target tissue is cut away or destroyed along with any commingled host tissue, with an effect similar to the use of a surgical instrument employing a cutting blade. Host tissue is inevitably eliminated and patient recovery time is extended. With thermolysis, cell integrity is damaged, potentially releasing harmful substances into surrounding tissues, leading to localized inflammation and damage to host cells, even necrotic tissue.

Selective photo-thermolysis avoids generalized application of thermal effects. Instead, thermal effects are modeled and confined to that specific and limited set of effects that lead to apoptosis. Predicting the thermal response can be modeled for the temperature distribution inside the tissue. The reaction with a target molecule can be considered as a two step process:

a) absorption of a photon promotes the molecule to an excited state; and

b) inelastic collisions with a molecule of the surrounding medium that leads to a deactivation and a simultaneous increase in the kinetic energy—therefore, the temperature rise microscopically originates from the transfer of photon energy to kinetic energy.

The effect of heat applied to cells ranges from temporary increases in temperature with no other effects, to coagulation, to vaporization, to complete carbonization with many other intermediate effects, named and unnamed. The temperature of exposure has an inverse relationship with the time required at this temperature to accomplish the desired effect. In addition, different wavelengths of light are absorbed differently with varying effects.

General biological effects related to different temperatures inside the tissue can be complex and are dependent on the type of tissue and laser parameters chosen. The most important and significant tissue alterations are thermal and chemical and attributed to conformational changes of molecules. These effects, accompanied by bond destruction and membrane alterations, are summarized in the single term hyperthermia, which is associated with a temperature ranging from approximately 42-50° C. Typically, a significant percentage of the tissue will die. Beyond 50° C., a measurable reduction in enzyme activity is observed, resulting in reduced energy transfer within the cell and immobility of the cell. Furthermore, certain repair mechanisms of the cell are disabled. Thereby, the fraction of surviving cells is further reduced. At 60° C., denaturation of proteins and collagen occurs which leads to coagulation of tissue and necrosis of cells. The corresponding macroscopic response is visible paling of the tissue. Several treatment techniques such as laser-induced interstitial thermotherapy aim at temperatures just above 60° C. At even higher temperatures (>80° C.), the membrane permeability is drastically increased, thereby destroying the otherwise maintained equilibrium of chemical concentrations. At 100° C. vaporization occurs.

In some embodiments, heat transfer is avoided where possible. Heat transfer is the product of heat generation and heat transport. Heat generation comprises parameters and optical tissue properties, primarily irradiance, exposure time, and the absorption coefficient with the absorption coefficient itself being a function of the laser wavelength. Heat transport comprises thermal tissue properties such as heat conductivity and heat capacity. Heat conduction is the primary mechanism of heat loss and heat transfer to non targeted tissue; heat flows are proportional to the temperature gradient within the system and resemble general diffusion rates.

In some embodiments, selective photo-apoptosis adjusts the duration of the laser pulse in order to minimize thermal damage to host cells. The scaling parameter for this time-dependent problem is the so-called thermal relaxation time. The two primary characteristics for optimization are duration and repetition rate.

Individual laser pulses with durations τ<1 μs typically do not produce thermal damage, which is sometimes referred to as the “1 μs rule”. Thermal effects do occur, however, from multiple laser pulses if the repetition rate is high enough to cause localized thermal accumulation. During the laser pulse, the temperature of the treatment area generally increases at a rate faster than homogeneous heat conduction can lower it. After the pulse ends, temperature decreases at a rate depending on conduction and other heat transport factors. If the next laser pulse is initiated before the thermal effects of the last pulse can fully dissipate, the treatment area temperature will rise. Adjacent tissue may also see an increase in temperature from heat conduction. If the treatment area includes both target and host tissue, different absorption characteristics of each tissue can lead to differences in thermal absorption, and therefore temperature, in each tissue from the same treatment. If a desired effect requires achieving a certain temperature in the target tissue and avoiding achieving the same or a different temperature in the host tissue, laser pulse characteristics, including wavelength and repetition, can be chosen to take advantage of these differences and achieve the desired results.

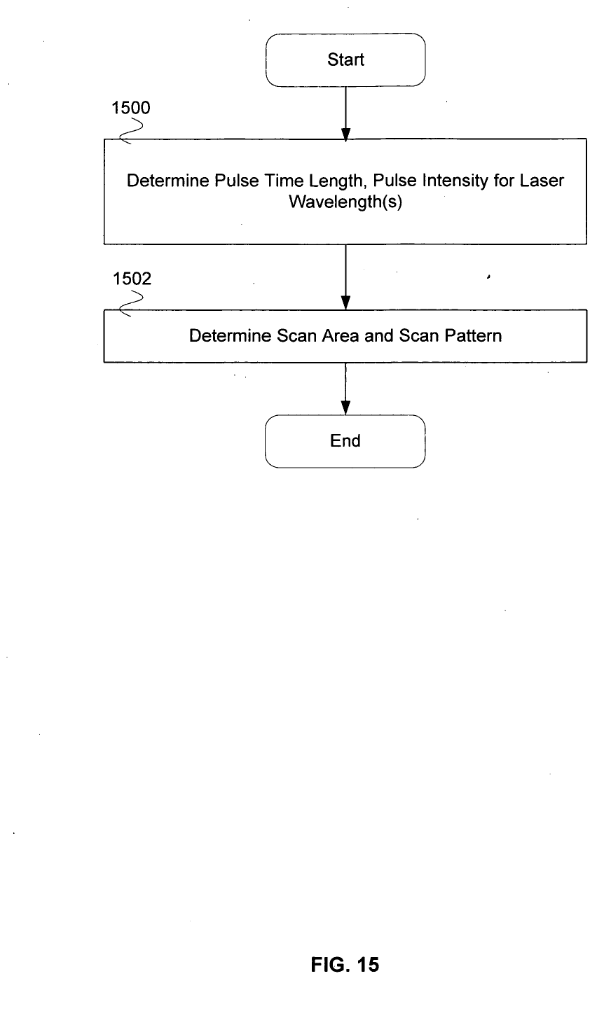

FIG. 15 is a flow diagram illustrating an embodiment of a process for determining a selective photo-apoptosis treatment. In various embodiments, the process of FIG. 15 is used to implement 1202 of FIG. 12, 1306 of FIG. 13, or 1402 of FIG. 14. In the example shown, in 1500 pulse time length and pulse intensity for one or multiple laser wavelengths are determined. For example, a series of picosecond long pulses (e.g., a burst of 20 pulses) of a fundamental and 2^{nd} overtone delivering a predetermined amount of light power are specified. In 1502, a scan area and a scan pattern are determined. For example, a circular pattern with pulses delivered every 45 degrees at 4 radii (e.g., 0.5 mm, 1 mm, 1.5 mm and 2 mm) where the outer edge of the treatment area receives less power to prevent neighboring host cells/tissue to remain undamaged.

Achieving the maximum therapeutic effect in a selective photo-apoptosis treatment requires the precise delivery of light energy with specific characteristics. Those specific characteristics are selected to simultaneously: a) achieve the desired effect of starting an apoptotic cascade within a targeted cell without lysing, ablating, or vaporizing that cell, and b) minimize the effect on surrounding host cells, either through the direct absorption of energy or through inter-cellular energy transfer. The required light energy and energy delivery can be best described by a combination of: 1) pulse characteristics, 2) pulse delivery pattern (pulse train), and 3) background energy.

Pulse characteristics are described by: a) light energy wavelength, b) pulse intensity, and c) pulse duration. The light energy wavelength is within the range of 300 nanometers to 20,000 nm, with greatest effectiveness in the range of 400 nm to 2,500 nm. Initial therapies are focused on the range of 650 nm and 1600 nm. Pulse intensity can be measured by average power over time or by peak power. While average power will typically be below, and often well below, 100 watts, peak power could be many orders of magnitude higher as pulses become shorter. Pulse duration are in the range of 1 femtosecond to 1 millisecond. Each pulse is generally delivered as part of a series of pulses. All of the pulses in this pulse delivery pattern, or pulse train, are identical or employ a variety of pulse characteristics. That series of pulses is further described as targeting an area equal to the size of the delivered energy pulse spot or targeting an area larger than one energy pulse spot.

When targeting an area larger than one energy pulse spot, one or more pulses are targeted at one spot and then one or more pulses are targeted at a different spot. It is customary to choose the next spot using a raster scan pattern that targets adjacent areas in a line until the edge of the desired treatment area is reached at which time the targeted area moves to the next line. In some embodiments, a pulse delivery pattern is employed that determines the next targeted spot using a statistical method pattern. The intent of the statistical method pattern is to minimize inter-cellular heat transfer by targeting non-adjacent spots yet still achieving uniform coverage. Each area is targeted once or more than once with the targeting patterned such that the entire treatment area receives a substantially uniform energy dose.

For example, a desired treatment area contains a number of targets each equal to the size affected by one energy pulse spot. The initial target is determined definitively or by using a statistical method. A treatment is applied and the location of the first target is recorded. All possible second targets are determined by selecting only targets that are neither the first target nor adjacent to the first target. The second target is selected from among the possible targets either with an algorithm or statistically. The treatment is applied to the second target and recorded. This process is repeated for each subsequent treatment: all possible targets are determined by eliminating those that have already received a treatment and those adjacent to one or more targets most recently treated. A target is selected and a treatment applied and recorded. This means the number of possible targets is reduced as more areas are treated. The last treatment is applied to the last target still untreated.

In some embodiments, it is desired that each target receive two or more treatments. All targets receive one treatment before any target receives a second or subsequent treatment. In some embodiments, the inclusion of an area in the list of possible targets uses the information of whether an area has already received fewer than the total desired number of treatments and if the previous treatment was in the immediately preceding or recent rounds. In some embodiments, for treatment at a specified depth or at various depths below a surface—for example, with multiple targets effectively stacked on top of each other—the target areas are described as a vector and the information determining inclusion in the list of subsequent targets includes target depth.

In some embodiments, therapeutic effect of selective photo-apoptosis is enhanced by the use of a general background radiation that brings target cells closer to a desired energy threshold. This background energy is either coherent non-coherent radiation in the range of 0.2 to 20 microns.

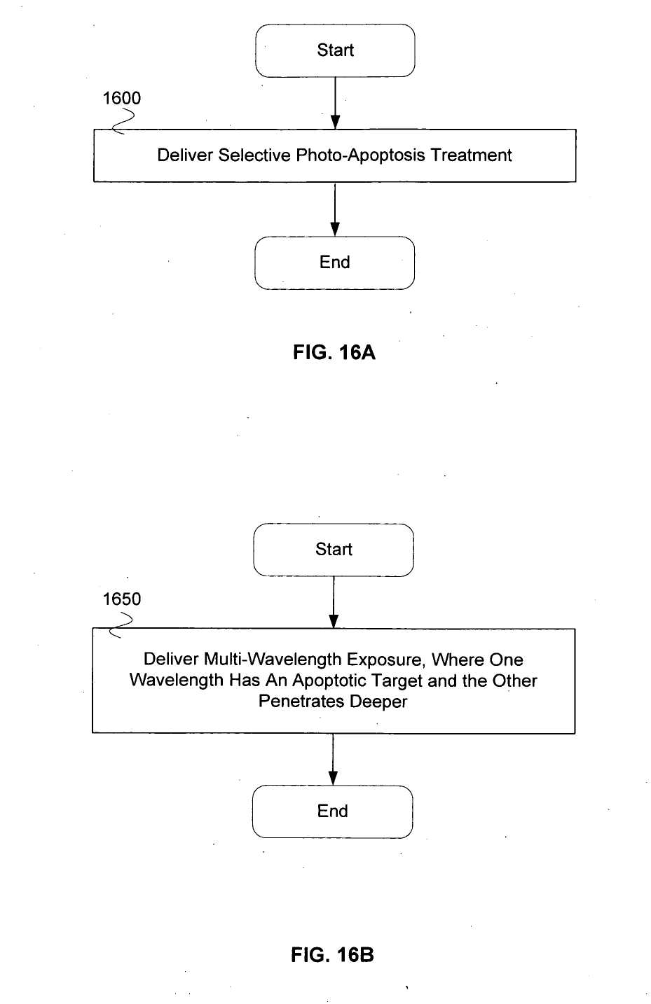

FIGS. 16A and 16B are flow diagrams illustrating embodiments of processes for a system for selective photo-apoptosis cancer treatment. In some embodiments, the processes of FIGS. 16A and 16B are executed using controller 500 of FIG. 5. In the example shown in FIG. 16A, in 1600 a selective photo-apoptosis treatment is delivered. In the example shown in FIG. 16B, in 1550 a multi-wavelength exposure is delivered, where one wavelength has been determined to be effective in delivering a selective photo-apoptosis treatment but cannot effectively penetrate to all desired treatment depths. In this example, the second wavelength penetrates more deeply into tissue but is not as effective in delivering a selective photo-apoptosis treatment. The second wavelength adds energy to the target types, reducing the amount of additional energy required to provide a selective photo-apoptosis treatment, and increasing the depth at which the first wavelength can remain effective in delivering a selective photo-apoptosis treatment. In various embodiments, the two wavelengths are a fundamental absorption wavelength and a related overtone, target multiple absorption bonds, are used to increase the effectiveness of a selective photo-apoptosis treatment without regard to penetration depth, are equally capable of delivering a selective photo-apoptosis treatment, are three or more wavelengths, may include black body radiation as one or more of the wavelengths, or are any other appropriate characteristics and combinations that enhance selective photo-apoptosis.

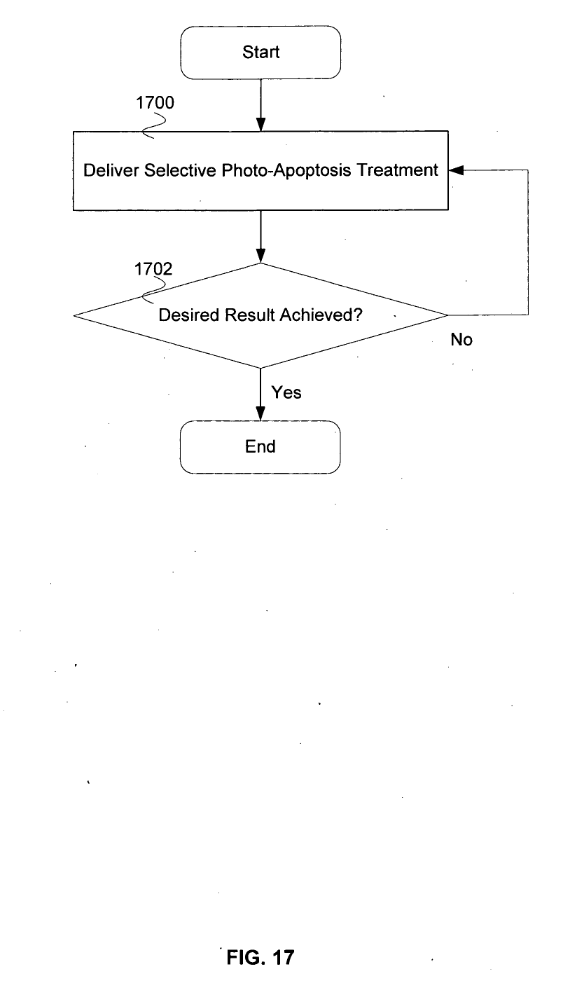

FIG. 17 is a flow diagram illustrating an embodiment of a process for a system for selective photo-apoptosis cancer treatment. In some embodiments, the process of FIG. 17 is implemented using controller 500 of FIG. 5. In the example shown, in 1700 a photo-apoptosis treatment is delivered. In 1702, it is determined whether a desired result has been achieved. In the event that a desired result has not been achieved, control passes to 1700. In the event that a desired result has been achieved, the process ends.

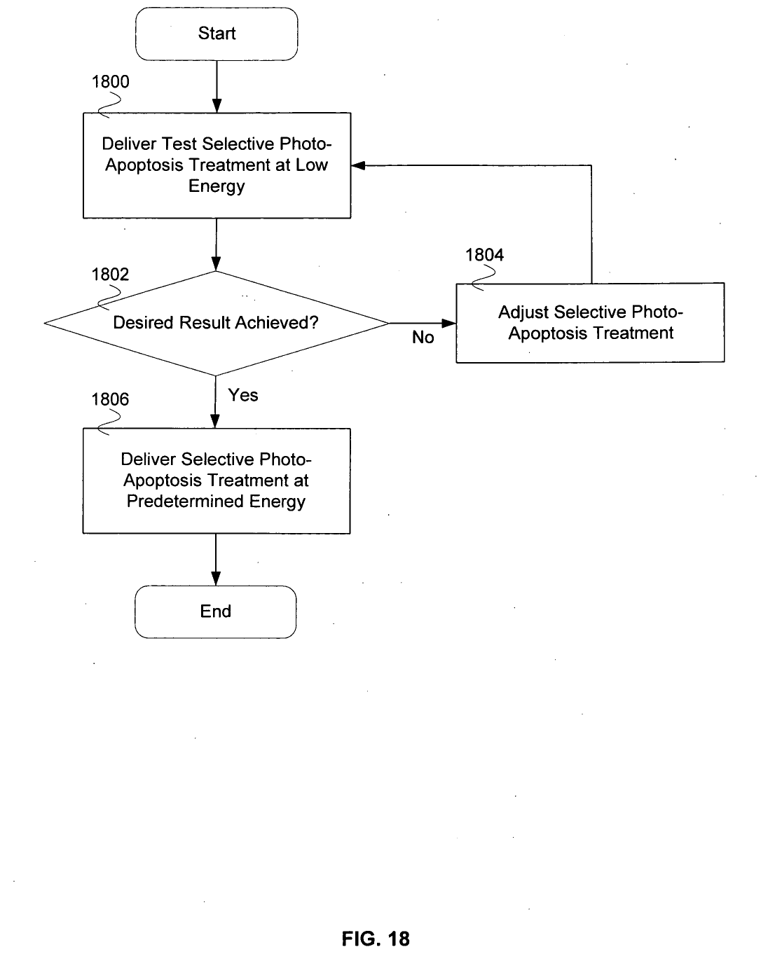

FIG. 18 is a flow diagram illustrating an embodiment of a process for a system for selective cancer treatment. In some embodiments, the process of FIG. 18 is implemented using controller 500 of FIG. 5. In the example shown, in 1800 a test selective photo-apoptosis treatment is delivered that may or may not be at low energy. In 1802, it is determined whether a desired result has been achieved. In the event that a desired result has not been achieved, in 1804 the selective photo-apoptosis treatment is adjusted and control passes to 1800. In the event that a desired result has been achieved, the photo-apoptosis treatment is delivered at the energy levels that have been thus determined. The treatment may be adjusted by location relative to host type tissue or to a tumor body or accumulation.

In some embodiments, the effectiveness of the described therapies are verified by discerning apoptosis from necrosis using biological markers and observations in a manner consistent with peer reviewed techniques. The verification using markers and observations are used to refine and verify the effectiveness of the selective treatment protocols for the various models studied. A large number of methods devoted to the identification of apoptotic cells and the analysis of the morphological, biochemical, and molecular changes that take place during this universal biological process have been developed including: a) mitochondrial and adenosine triphosphate/adenosine diphosphate (ATP/ADP) assays that give an early indication of the initiation of cellular apoptosis by the collapse of the electrochemical gradient across the mitochondrial membrane and the release of cytochrome C; b) assays using annexin V that binds to phosphatidylserine which is translocated to the outer surface of the cell early in the apoptotic process; c) caspase related assays that measure caspase activity because caspases are activated during apoptosis; and d) deoxyribonucleic acid (DNA) assays that measure generation of DNA fragments created during the execution phase of apoptosis.

Although the foregoing embodiments have been described in some detail for purposes of clarity of understanding, the invention is not limited to the details provided. There are many alternative ways of implementing the invention. The disclosed embodiments are illustrative and not restrictive.

What is claimed is:

1. A system for cancer treatment, comprising:
a processor configured to receive a target type and a host type and determine one or more illumination source characteristics such that:
an illumination of the target type employing the one or more illumination source characteristics induces apoptosis in the target type without initiating thermolysis or ablation of the target type; and
an illumination of the host type employing the one or more illumination source characteristics does not substantially induce apoptosis, thermolysis, or ablation in the host type; and
a memory coupled to the processor and configured to provide the processor with instructions.

2. A system as in claim 1, wherein the illumination of the target type targets a desired penetration depth.

3. As system as in claim 1, wherein the illumination source characteristics include an illumination source having at least two wavelengths.

4. A system as in claim 3, wherein the at least two wavelengths comprise a fundamental absorption wavelength and a related overtone.

5. A system as in claim 3, wherein the at least two wavelengths are used in combination to achieve a desired penetration depth.

6. A system as in claim 3, wherein the at least two wavelengths target multiple absorption bonds of the target type.

7. A system as in claim 1, wherein the illumination of the target type uses a pattern for scanning an area larger than an area illuminated by the illumination source.

8. A system as in claim 7, wherein the pattern comprises one or more of the following: a statistical pattern that ensures complete coverage of the area while avoiding illuminating an area adjacent to the target type, a change pattern that includes a scan rate change, or a skipping pattern.

9. A system as in claim 7, wherein the pattern comprises a time varied series of pulses of the illumination.

10. A system as in claim 1, wherein a selective treatment is determined based at least in part on a measurement using a low power test treatment.

11. A system as in claim 1, further comprising a processor configured to determine a characteristic of the target type based on a measurement made after a selective treatment.

12. A system as in claim 1, wherein one of the one or more illumination source characteristics comprises a background illumination.

13. A method for cancer treatment, comprising:
receiving a target type and a host type; and
determining one or more illumination source characteristics such that:
an illumination of the target type employing the one or more illumination source characteristics induces apoptosis in the target type without initiating thermolysis or ablation of the target type; and
an illumination of the host type employing the one or more illumination source characteristics does not substantially induce apoptosis, thermolysis or ablation in the host type.

14. A computer program product for cancer treatment, the computer program product being embodied in a computer readable storage medium and comprising computer instructions for:
receiving a target type and a host type; and
determining one or more illumination source characteristics such that:
an illumination of the target type employing the one or more illumination source characteristics induces apoptosis in the target type without initiating thermolysis or ablation of the target type; and
an illumination of the host type employing the one or more illumination source characteristics does not substantially induce apoptosis, thermolysis, or ablation in the host type.
